(19) United States
(12) Patent Application Publication
(10) Pub. No.: US 2008/0085211 A1
(43) Pub. Date: Apr. 10, 2008

(54) Method for Sterilizing Biological Materials

(75) Inventor: Lynn L.H. Huang, Yongkang City (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: NATIONAL CHENG KUNG UNIVERSITY, Tainan City (TW)

(21) Appl. No.: 11/866,564

(22) Filed: Oct. 3, 2007

(30) Foreign Application Priority Data
Oct. 4, 2006 (TW) 95136924

(51) Int. Cl. A61L 2/20

(52) U.S. Cl. 422/28

(57) ABSTRACT

A method for sterilizing biological materials is disclosed. After dehydrating the biological material, the dehydrated biological material is put into a closed container, and 0.5 ppm to 100 ppm of ozone gas is introduced into the container for a period of time until the biological material is completely sterilized. Afterward, the ozone gas is removed from the closed container to finish sterilization of the biological material.

### RELATED APPLICATIONS

This application claims priority to Taiwan Application Serial Number 95136924, filed Oct. 4, 2006, which is herein incorporated by reference.

### BACKGROUND

1. Field of Invention

The present invention relates to a sterilizing method. More particularly, the present invention relates to a method for sterilizing biological materials employing ozone.

2. Description of Related Art

Typically, biological materials, which may refer to materials existing in or derived from living organisms, substantially comprise components, such as amino acids, peptides, proteins, polysaccharides and so on, directly extracted from microorganisms, animals or plants. As the biological material itself possesses excellent biocompatibility, it has potential in medical applications, for example, wound dressing and scaffold for tissue engineering, as well as in pharmaceutical and cosmetic industries. The biological materials for human or living organisms must be subjected to a strictly sterilizing procedure. However, most of the biological materials are susceptible to high temperature sterilization, and they are also liable to be denatured. The option of methods for sterilizing the biological materials is very restricted. Thus, the application of the biological materials is presently focused on how to achieve the sterilizing effect and to save the bioactivity of biological materials, rather than destroying their properties.

There are several methods of sterilizing biological materials as follows. (1) Sterilization with 75% ethanol: The biological material is immersed in 75% ethanol, and it must be reserved and delivered in moist state. However, the bioactive components are liable to be denatured in such moist state. Moreover, it is not sure whether ethanol is completely removed from or remains in the biological material when rinsing it before use. (2) Sterilization with gamma (Υ)-irradiation as disclosed in U.S. Pat. No. 5,485,496, and Taiwan Pat. Nos. 145,942 and 115,972: This method is applied commonly, which employs Υ-ray to irradiate the biological materials. However, the energy of the Υ-ray is so high that some chemical structures of the biological material are destroyed, resulting in weakening the mechanical strength of the biological material. In addition, the irradiation is hazardous to human so that it has to be operated in a specific place, resulting in inconvenient usage. (3) Sterilization with ultraviolet light as disclosed in Taiwan Pat. No. 474,828: This method employs ultraviolet light to irradiate the biological materials for sterilization. Nevertheless, the ultraviolet light penetrates to minimal distances and only the surfaces of the biological materials can be sterilized. Thus, the ultraviolet light is unavailable to sterilize the biological materials mostly with three-dimensional shape and opaque property. (4) Sterilization with chemical reagents as disclosed in U.S. Pat. Nos. 5,460,962 and 6,096,266, as well as Taiwan Pat. Nos. 310,308, 241,193 and 149,465: This method is accomplished by adding chemical bactericides into the biological materials. However, the chemical bactericides are toxic and difficult to be removed, so it is applied in fewer fields. (5) Sterilization with high temperature and high pressure (autoclave) as disclosed in Taiwan Pat. No. 443,932: The autoclave method results in the denaturation of biological materials, even completely losing their bioactivity. In sum, the aforementioned methods have respective drawbacks, which often cause biological materials to change in chemical structures and properties, resulting in biocompatibility and applicability.

Ozone is typically applied in surface modification of polymeric biomaterials. Ozonization refers to generate activated peroxide on the surface of the biomaterial, and it further induces graft copolymerization with some functional groups on the biomaterial, as well as degradation in aqueous environment. For the use of sterilization, ozone is usually applied in sterilization of general instruments as disclosed in U.S. Pat. No. 5,788,941 and Taiwan Pat. No. 061,995. This method is accomplished by placing the object into an ozone-containing environment. In general, a biological material has certain aqueous content and even exists in a solution state. As such for the aqueous biological material, the aqueous content existing in the sample may react with ozone gas, resulting in changes of chemical functional groups inside the biological material, and even micro-changes inside the structures of the biological material, such as polymerization, degradation and so on, thereby affecting physiochemical properties of the biological material. On the other hand, as such for the biological material in solution state, ozone dissolved in the solution may be insufficient to achieve a desirable sterilizing effect. If ozone is directly introduced into an aqueous solution, the same problem caused by ozone sterilization to the water-containing biological material will happen.

For the foregoing reasons, it is necessary to develop a method for sterilizing biological materials, while maintaining bioactivity and structure thereof, such that the method can be widely applied to the biological materials.

### SUMMARY

The present invention develops a novel method for sterilizing biological materials, which can overcome the shortcoming of the biological materials that those structure can be destroyed by ozone in the prior art, and further apply ozone to sterilize the biological materials.

According to one embodiment of the present invention, a method for sterilizing biological materials is provided as follows. After dehydrating the biological material, the dehydrated biological material is put into a closed container, and 0.5 ppm (parts per million) to 100 ppm of ozone gas is introduced into the container for a period of time until the biological material is completely sterilized. Afterward, the ozone gas is removed from the closed container to finish sterilization of the biological material.

According to another embodiment of the present invention, a method for sterilizing collagen is provided as follows. After dehydrating the collagen, the dehydrated collagen is put into a closed container, and 0.5 ppm to 100 ppm of ozone gas is introduced into the container for a period of time until the collagen is completely sterilized. Afterward, the ozone gas is removed from the closed container to finish sterilization of the collagen.

### BRIEF DESCRIPTION OF THE DRAWINGS

The foregoing aspects and many of the attendant advantages of this invention are more readily appreciated and better understood by referencing the following detailed description, when taken in conjunction with the accompanying drawings, wherein:

FIG. 1 is a micrograph at 100× magnification showing fibroblast morphology according to an embodiment of the present invention, where the fibroblasts were cultured on the collagen matrix sterilized by the present method;

FIG. 2 is a photo of culture tubes wherein staphylococci were cultured in LB broth added with the unsterilized collagen matrix, the ozone sterilized collagen matrix, unsterilized collagen solution, and the ozone sterilized collagen solution, from left to right; after 16-hour incubation, those cultures are observed in turbidity; and

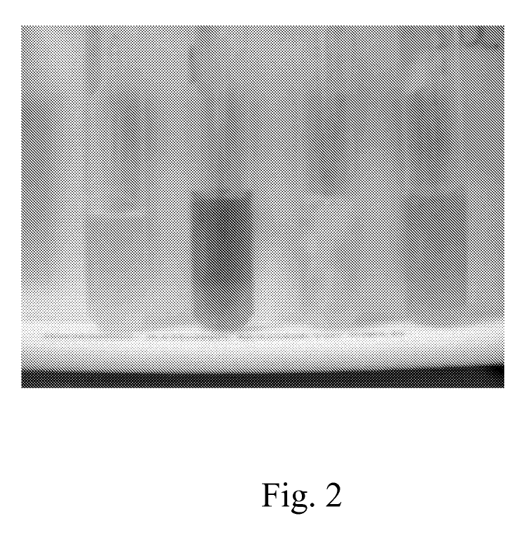

FIG. 3 is a stained electrophoresis gel of collagen subjected to the following respective treatments: (1) untreatment; (2) treatment with ozone gas as EXAMPLE 1; (3) treatment with ultraviolet irradiation for 12 hours; (4) immersion in 75 vol. % ethanol for 4 hours; (5) immersion in 2 vol. % formaldehyde for 1 hour; or (6) autoclave sterilization under conventional high-temperature and high-pressure.

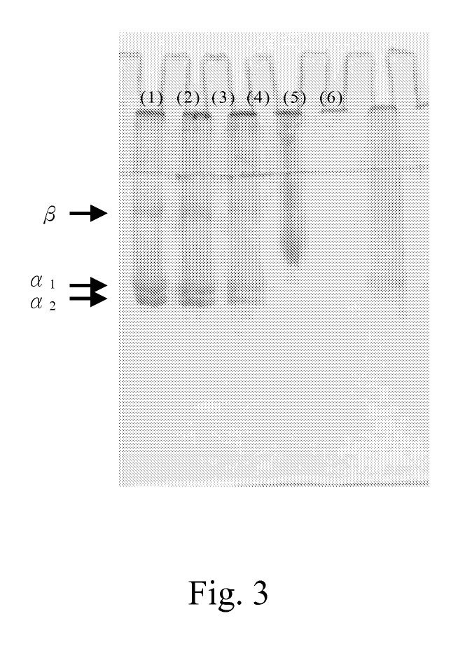

### DESCRIPTION OF THE PREFERRED EMBODIMENTS

The below description providing various embodiments and specific details of the invention is illustrative only and should not be construed in any way as limiting the invention. Furthermore, various applications of the invention, and modifications thereto, which may occur to those who are skilled in the art, are also encompassed by the general concepts described below.

The present invention provides a method for sterilizing biological materials. In one embodiment, the method for sterilizing biological materials is performed as follows. After dehydrating the biological material, the dehydrated biological material is put into a closed container, and 0.5 ppm to 100 ppm of ozone gas is introduced into the container for a period of time until the biological material is completely sterilized. Afterward, the ozone gas is removed from the closed container to finish sterilization of the biological material.

“The biological material” in the specification refers to a material existing in a living organism, produced by a living organism or for use in a living organism. A preferred embodiment of the biological material of the present invention is a material, for example, growth factor, antibody, hormone, protein drug, collagen, gelatin, saccharide, oligosaccharide, polysaccharide, hyaluronan, elastin, chondroitin sulfate, heparin, heparin sulfate, dermatan sulfate, glycosaminoglycan, chitin, chitosan, alginate or related derivatives, existing in a living organism. However, collagen is more preferable among those. Moreover, the biological material of the present invention may be also a material, for example, including an enzyme, a protein product, a protein drug, a cell culture material with a biological component, a matrix of artificial tissue and organ, a genetic-engineering product, a material of Chinese herb medicine, a product of Chinese herb medicine, a cosmetic product and a cosmetic additive, produced by a living organism. Furthermore, the biological material of the present invention may be a material, for example, including a cell culture material with a biological component and a matrix of artificial tissue and organ, for use in a living organism.

The present invention is characterized by removing the water from the biological material, so as to prevent the shortcomings in the prior art, such as undesired reaction between ozone and water, or the insufficient content of ozone in the water. Even though one skilled in the art commonly knows the methods and conditions how to remove the water, the present method can remove water, rather than substantially affect inherent bioactivities and physiochemical properties of the biological materials. According to an embodiment of the present invention, the aforementioned step of dehydrating the biological materials may utilize lyophilization. According to another embodiment of the present invention, the aforementioned step of dehydrating the biological materials may dry under low temperature and decreased pressure.

According to the present method, the dehydrated biological material is put into a closed container for sterilization with ozone. The closed container of the present invention is suitable for receiving the biological material therein, and it is beneficial to supply ozone gas therein or exhaust ozone gas therefrom. Preferably, the closed container has a channel for supplying and exhausting ozone, so as to control ozone in and out.

The concentration of ozone depends on the quantity and property of the biological material. In general, the ozone concentration is in a range from 0.5 ppm to 100 ppm, and preferably, from 1 ppm to 50 ppm. The period of sterilization time of ozone also depends on the quantity and property of the biological material until the biological material is completely sterilized. For example, 30 minutes may be needed for sterilizing the biological material of collagen.

According to the present method, the ozone gas is then removed from the closed container. One skilled in the art commonly knows the methods how to remove the ozone gas. In a preferred embodiment of the present invention, the ozone gas may be removed in the manner of vacuuming removal, sterile gas exchange removal or standing removal.

According to the present method, after dehydrating the biological material, the chemical structure of the dehydrated biological material sterilized by ozone is destroyed far less than that sterilized by gamma (Υ)-irradiation, and the biological material is neither degraded nor polymerized. Moreover, the biological material sterilized by ozone is safer than that sterilized by radioactive rays, and the ozone sterilization is conveniently applied anywhere rather than in specific place. As such, it is not worried about any irritant substance remaining in the biological material in comparison to that treated by chemical cross-linking agents. Besides, the ozone sterilization is beneficial to retain inherent properties of biological materials, to preserve and to transport biological materials conveniently.

The present invention further provides another method for sterilizing collagen. In one embodiment, the method for sterilizing collagen is performed as follows. After dehydrating collagen, the dehydrated collagen is put into a closed container, and 0.5 ppm to 100 ppm of ozone gas is introduced into the container for a period of time until the collagen is completely sterilized. Afterward, the ozone gas is removed from the closed container to finish sterilization of the collagen.

The following embodiments of the present invention are described in detail as an illustration of the present invention rather than a limitation thereof.

### EXAMPLE 1

### Sterilization of Collagen

EXAMPLE 1 is described with respect to sterilization of the collagen solution. The collagen solution is lyophilized to dehydrate collagen molecules. And then, the dehydrated collagen is put into a closed container of 21 cm (length)×15 cm (width)×7 cm (height), and 120 mg/hour (approximately 27.2 ppm) of ozone gas is introduced into the container for approximate 30 minutes. The ozone gas remained in the collagen is then removed by standing at ventilated laminar flow stage for about 1 hour at room temperature, or alternatively, vacuuming for 1 hour, so as to finish sterilization of the biological material.

### EXAMPLE 2

### Effect of Ozone Sterilization of Collagen

EXAMPLE 2 is described with respect to sterilization effect on the collagen sterilized by EXAMPLE 1 as experiment, compared with that sterilized by conventionally ultra-high-speed centrifugation as comparison and/or that without sterilization as control.

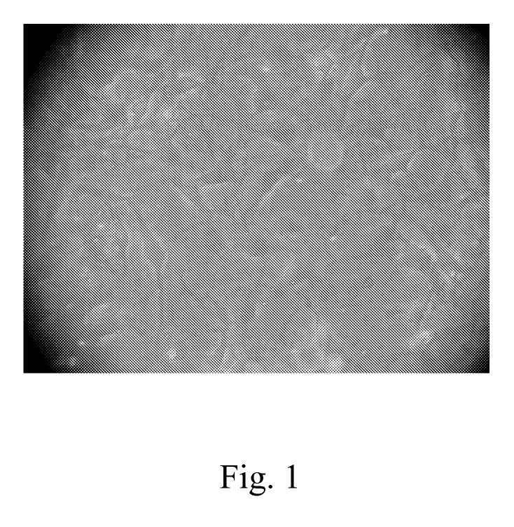

Cell morphology: The human foreskin fibroblasts were seeded on the collagen matrices, which were sterilized by EXAMPLE 1 or conventional ultra-high-speed centrifugation, respectively, and their cell morphologies were observed under light microscope and photoed under 100× magnification as shown in FIG. 1. Reference is made to FIG. 1, where the fibroblasts grown on the collagen matrix sterilized by ozone gas are similar to those grown on the collagen matrix sterilized by conventionally ultra-high-speed centrifugation (unshown).

Total cell numbers: The human foreskin fibroblasts were seeded on either the collagen matrix or with collagen solution sterilized by EXAMPLE 1 or conventional centrifugation, respectively. Dulbecco's Modified Eagle's Medium (DMEM) supplemented with 10 vol. % fetal bovine serum (FBS) were used to culture fibroblasts. After a period of incubation, the collagen matrix was digested by collagenase, and the total cell numbers were then counted and the percentages of cell numbers in comparison to the control were listed as the following Table 1.

TABLE 1

| Treatment of Collagen | Total Cell Number (%) |
|---|---|
| Collagen solution sterilized by | 100 ± 4.3 |
| conventional centrifugation | |
| Collagen solution sterilized by | 96 ± 4.3 |
| ozone gas | |
| Collagen matrix sterilized by | 100 ± 3.4 |
| conventional centrifugation | |
| Collagen matrix sterilized by | 106 ± 8.6 |
| ozone gas | |

The result indicates that the growth of fibroblasts cultured either on the collagen matrix or with collagen solution, sterilized by EXAMPLE 1 or conventional centrifugation, was very similar.

Integrity of collagen molecule: The molecular integrity of collagen, which were collagen solution or the dehydrated collagen matrix, unsterilized or

What is claimed is:

1. A method for sterilizing a biological material, comprising:
dehydrating the biological material;
putting the dehydrated biological material into a closed container, and introducing 0.5 ppm to 100 ppm of ozone gas into the container for a period of time until the biological material is completely sterilized; and
removing the ozone gas from the closed container to finish sterilization of the biological material,
wherein the biological material substantially remain intact after having been subjected to the sterilization.

2. The method of claim 1, wherein the biological material is selected from the group consisting of growth factor, antibody, hormone, protein drug, collagen, gelatin, saccharide, oligosaccharide, polysaccharide, hyaluronan, elastin, chondroitin sulfate, heparin, heparan sulfate, dermatan sulfate, glycosaminoglycan, chitin, chitosan, alginate and derivatives thereof.

3. The method of claim 2, wherein the biological material is collagen or derivatives thereof.

4. The method of claim 1, wherein the biological material is a material produced by a living organism.

5. The method of claim 1, wherein the biological material is selected from the group consisting of an enzyme, a protein product, a protein drug, a cell culture material with a biological component, a matrix for creating artificial tissue or organ, a genetic-engineering product, a material of Chinese herb medicine, a product of Chinese herb medicine, a cosmetic product, a cosmetic additive, a growth factor, an antibody, a hormone, collagen, gelatin, saccharide, oligosaccharide, polysaccharide, hyaluronan, elastin, chondroitin sulfate, heparin, heparan sulfate, dermatan sulfate, glycosaminoglycan, chitin, chitosan, alginate and related derivatives thereof.

6. The method of claim 1, wherein the biological material is a material for use in a living organism.

7. The method of claim 6, wherein the biological material is selected from the group consisting of a cell culture material with a biological component, a matrix for creating an artificial tissue or an organ, growth factor, antibody, hormone, protein drug, collagen, gelatin, saccharide, oligosaccharide, polysaccharide, hyaluronan, elastin, chondroitin sulfate, heparin, heparan sulfate, dermatan sulfate, glycosaminoglycan, chitin, chitosan, alginate and related derivatives thereof.

8. The method of claim 1, wherein the step of dehydrating the biological material is carried out by lyophilization.

9. The method of claim 1, wherein the step of dehydrating the biological material is carried out by drying under low temperature and decreased pressure.

10. The method of claim 1, wherein the concentration of ozone gas is between about 1 ppm and about 50 ppm.

11. The method of claim 1, wherein the step of removing the ozone gas is carried out by utilizing vacuum degassing, sterile gas exchange or standing degassing.

12. A method for sterilizing collagen, comprising:
dehydrating collagen;
putting the dehydrated collagen into a closed container, and introducing 0.5 ppm to 100 ppm of ozone gas into the container for a period of time until the collagen is completely sterilized; and
removing the ozone gas from the closed container to finish sterilization of the collagen,
wherein the collagen substantially remain intact after having been subjected to the sterilization.

13. The method of claim 12, wherein the step of dehydrating the collagen is carried out by lyophilization.

14. The method of claim 12, wherein the step of dehydrating the collagen is carried out by drying under low temperature and decreased pressure.

15. The method of claim 12, wherein the ozone gas is introduced in a range of 1 ppm to 50 ppm.

16. The method of claim 12, wherein the step of removing the ozone gas is carried out by utilizing vacuuming degassing, sterile gas exchange or standing removal.

17. A method for sterilizing a dehydrated biological material, comprising:
putting the dehydrated biological material into a closed container, and introducing 0.5 ppm to 100 ppm of ozone gas into the container for a period of time until the biological material is completely sterilized; and
removing the ozone gas from the closed container to finish sterilization of the biological material,
wherein the biological material substantially remain intact after having been subjected to the sterilization.

18. The method of claim 17, wherein the biological material is selected from the group consisting of a cell culture material with a biological component, a matrix for creating an artificial tissue or an organ, an enzyme, a protein product, a protein drug, a cell culture material with a biological component, a matrix of artificial tissue and organ, a genetic-engineering product, a material of Chinese herb medicine, a product of Chinese herb medicine, a cosmetic product, a cosmetic additive, growth factor, antibody, hormone, protein drug, collagen, gelatin, saccharide, oligosaccharide, polysaccharide, hyaluronan, elastin, chondroitin sulfate, heparin, heparan sulfate, dermatan sulfate, glycosaminoglycan, chitin, chitosan, alginate and derivatives thereof.

19. The method of claim 17, wherein the biological material is collagen or derivatives thereof.
